(19) United States
(12) Patent Application Publication
(10) Pub. No.: US 2024/0225521 A9
(43) Pub. Date: Jul. 11, 2024

(54) APPARATUS FOR AND METHOD OF MEASURING INTRACRANIAL DYNAMICS

(71) Applicant: OULUN YLIOPISTO, Oulu (FI)

(72) Inventors: Teemu MYLLYLÄ, Oulu (FI); Vesa KIVINIEMI, Oulu (FI)

(21) Appl. No.: 18/547,440

(22) PCT Filed: Feb. 23, 2022
(86) PCT No.: PCT/FI2022/050119

§ 371 Date: Aug. 22, 2023

(30) Foreign Application Priority Data
Feb. 23, 2021 (FI) 20215198

(51) Int. Cl. A61B 5/00; A61B 5/03; A61B 5/053; A61B 5/369

(52) U.S. Cl. CPC A61B 5/4058; A61B 5/0075; A61B 5/0082; A61B 5/031; A61B 5/053; A61B 5/369

(57) ABSTRACT

An apparatus for measuring intracranial dynamics comprises the at least one sensing device (100): an electroencephalo-graphic electrode arrangement, which senses direct-current electroencephalographic signals from the brain, an optic measurement Marrangement (120), which directs optic radiation toward the brain through the cranium, and receives the optic radiation reflected and/or scattered therefrom, and/or a capacitive sensor arrangement (130), which senses electric potential signals of the head. The apparatus additionally comprises a data processing arrangement (150), which receives electric signals from the at least one sensing device (100), and determine data on at least one of the following dynamics: glymphatic activity, water within the cranium, brain tissue movements, water and/or electrolyte movements and intracranial pressure based on said electric signals from the at least one sensing device (100). The data processing arrangement (150) then outputs at least one piece of the data on the dynamics through a user interface (152).

### FIELD

The invention relates to an apparatus for and a method of measuring intracranial dynamics.

### BACKGROUND

Proper dynamics of a neurological central nervous system (CNS) is a function of the wellbeing of mammals such as a human beings. The CNS dynamics are to a large degree related to dynamic changes in electrolyte concentrations in various water compartments, in short brain electrohydrodynamics both inside and outside neurons in the CNS, and also as recently shown, to the intracranial water dynamics including the water in the brain tissue and in the free cerebrospinal fluid (CSF) in the intracranial spaces like aqueducts, perivascular spaces and ventricles. The dynamic interactions of the three main CNS water compartments; CSF cerebral blood and intracellular fluid in brain tissue can be considered closely related to the glymphatic function. The study of glymphatic water dynamics of CSF within the cranium is a fairly new branch within neurophysiology.

However, monitoring and measuring the dynamics within the cranium has been challenging. Currently, the glymphatic neurohydrodynamics can be directly assessed to certain extent by injecting tracers such as Gd3+ contrast media into human and other mammalian CSF and measuring the spread and removal of the tracers from the brain with several repeated magnetic resonance imaging (MRI) scans over one to two days in humans. This method requires resource intensive procedures such as invasive punctures of the CSF space with potential risks of infection, bleeding, headache and other more severe neurologic sequelae on the patient and requires too much scanning time for realistic patient monitoring of neurologic patients in clinical imaging settings.

Another even more invasive procedure administer gadolinium complexes Gd3+ as a contras media for the MRI scan inside brain tissue with focused ultrasound driven blood-brain-barrier (BBB) opening to show how the glymphatic neurohydrodynamics functions within a living human brain tissue to remove foreign contrast materials from the brain. Currently there are no monitoring devices for sleep-time or upright neurohydrodynamics. Hence, an improvement would be welcome.

### BRIEF DESCRIPTION

The present invention seeks to provide an improvement in the measurements.

The invention is defined by the independent claims. Embodiments are defined in the dependent claims.

### LIST OF DRAWINGS

Example embodiments of the present invention are described below, by way of example only, with reference to the accompanying drawings, in which

FIG. 1 illustrates an example of apparatus for measuring intracranial dynamics;

FIG. 2 illustrates an example of an optic measurement arrangement;

FIG. 3 illustrates an example of a capacitive sensor arrangement;

FIG. 4 illustrates an example of extinction coefficient of water, deoxyhemoglobin and oxyhemoglobin in a spectral band;

FIG. 5 illustrates an example of a neurovascular unit (blood vessel, perivascular space and interstitial brain tissue compartment);

FIG. 6 illustrates an example of direct current electroencephalogram (DC-EEG) and functional near-infrared spectroscopy (NIRS) data changes during therapeutic altering human blood-brain barrier permeability;

FIG. 7 illustrates an example of dynamics of the cerebral arterial blood and the cerebral venous blood during osmotic mannitol induced i.a. BBB opening;

FIG. 8 illustrates an example of human data determining glymphatic fluid dynamics based on inverse nature of brain water and cerebral blood hydrodynamics;

FIG. 9 illustrates an example of optically measured time series during visual tasks and a corresponding magnetic resonance encephalography (MREG) blood oxygen level dependent (BOLD) time series;

FIG. 10 illustrates an example of location of measurement and brain pulsation with respect to CSF movement;

FIG. 11 illustrates an example of a brain pulse waveform of a young person and decompositions;

FIG. 12 illustrates an example of a brain pulse waveform of an elder person and decompositions;

FIG. 13 illustrates an example of variation in recorded brain pulse waveforms at different wavelengths used in the analysis method;

FIG. 14 illustrates an example of recorded signals in time domain used in the analysis method;

FIG. 15 illustrates an example of the spectral powers the signals recorded from the brain and used in the analysis method;

FIG. 16 illustrates an example of a data processing unit; and

FIG. 17 illustrates of an example of a flow chart of a measuring method.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments to consist of only those features that have been mentioned and such embodiments may also contain features/structures that have not been specifically mentioned. All combinations of the embodiments are considered possible if their combination does not lead to structural or logical contradiction.

It should be noted that while Figures illustrate various embodiments, they are simplified diagrams that only show some structures and/or functional entities. The connections shown in the Figures may refer to logical or physical connections. It is apparent to a person skilled in the art that the described apparatus may also comprise other functions and structures than those described in Figures and text. It should be appreciated that details of some functions, structures, and the signalling used for measurement and/or controlling are irrelevant to the actual invention. Therefore, they need not be discussed in more detail here.

Recently described glymphatic brain clearance mechanism, which can be monitored using the apparatus for measuring intracranial dynamics described in this document, uses physiological and mechanical pulsations of the brain and the cerebrospinal fluid to convect waste and metabolites along paravascular space in the brain tissue. The glymphatic system seems to clear the brain especially during sleep, and an improper glymphatic convection may decrease wellbeing of a mammal such as a human being. Additionally, a failure of the glymphatic convection has even been connected to several major brain disorders including Alzheimer's disease, fronto-temporal dementia, vascular dementia, normal pressure hydrocephalus, multiple sclerosis, epilepsy, trauma, stroke, tumors, hydrocephalus, Chiari malformation, syringomyelia, pseudotumor cerebri, cerebral vasospasm, glaucoma, cerebral aneurysms or the like for example. Although the dynamics of the brain and water within the cranium can be measured and monitored using the apparatus described in this document, determination and/or diagnosis of any potential disease based on the measurement results expressly remains to a medical personnel. Namely, the measured dynamics may be based on the heart beat and/or the respiration and/or low frequency vasomotor waves (Mayer & Traube-Hering waves).

This document describes optics based measurement of volume dynamics of the brain and water within the cranium. The water within the cranium includes interstitial fluid, CSF and blood, which are all mainly water (>90%). The measurement may additionally or alternatively be combined with direct-current (DC) electroencephalography (EEG) and/or capacitance measurement, or the measurement may be performed alone as either the DC electroencephalography or the capacitance measurement. The study of glymphatic water dynamics of CSF within the cranium is a fairly new branch within neurophysiology and the optical measurement described in this document reveals the previously missing mechanism of brain clearance.

By using a light source with at least three wavelengths in near-infrared range (NIR) range (one below 800 nm, one between 800 nm and 940 nm and one above 940 nm, for example), for example, and at least one light detector for these wavelengths, brain tissue dynamics and/or water/cerebrospinal fluid (CSF) dynamics can be measured. Additionally, blood/hemoglobin dynamics may be measured. The brain tissue water dynamics can be seen as pulsatile flow movement, and is caused by mainly by three leading physiological pulsation sources: cardiorespiratory and vasomotor pulsations. Each of the pulsation source has a different, non-overlapping frequency range that can be used to identify the pulsation source. This further can be used to identify the measured CNS water compartments and importantly to identify failures of the glymphatic clearance mechanisms. In addition, movements or vibration of head can be used as a pulsation source. Different NIR-light wavelengths may result in specific intensity changes or pulse shapes of the detected light, and this shape can be used to both identify water compartment and to estimate continuously changes related to brain stiffness and intracranial pressure that may all affect the glymphatic activity.

Using direct current EEG (DC-EEG) arrangement with at least two electrodes, it is possible to measure electric potential between brain tissue interstitium and blood for direct information on glymphatic water/electrolyte permeable movement over in the glia limitans interface between blood and brain tissue, i.e. from the glymphatic paravascular space. Interaction between the gathered signals, in particular their specific phase differences, and amplitude changes reflect activation of the glymphatic system and blood-brain barrier permeability.

Currently, we are able to measure and analyse the mentioned signals and their interactions. The apparatus of this document may be wearable, and it can be considered a “Glymphometer” that can be used for long-term monitoring purposes of well-being.

Glymphatic brain CSF convection and clearance precedes several chronic brain diseases such as Alzheimer's disease, chronic traumatic encephalopathy, and in tumors and focal epilepsies scar formation prevents the normal CSF convection. After bleeding or arterial ischemic occlusion CSF (or brain water) may flows into the brain tissue inducing edema and increased pressure. The glymphatic system is similar in mammals including humans and mice as it has been shown to be similar with magnetic resonance imaging with invasive gadolinium agent (MRI Gd3⁺) intrathecal injections and contrast media imagings lasting 24-48 h. Currently there are no monitors or tools to non-invasively measure the glymphatic function. There are no solutions for non-invasive, easy to wear, bedside/overnight monitoring of glymphatic function during sleep. It is very important to monitor glymphatic function without affecting sleep quality. Also it is not known how the glymphatic clearance convection alters when the subject is in upright position.

What is presented in this document enables easy monitoring of sleep quality and can quantify the glymphatic clearance increase of sleep and importantly monitor the subject also in upright position in daily activity. Further, it provides a method to study and diagnose how, such as, physiological exercise and different treatments affect glymphatic clearance and wellbeing of the mammal such as a human being.

The monitoring may be performed during overnight sleep, which can easily be done with the wearable support structure 102 having at least sensing device 100. Additionally the measurement results may be possible to use diagnosing brain diseases such as Alzheimer's disease, stroke, Parkinson, epilepsy, tumors, etc. but that is the work of the medical personnel.

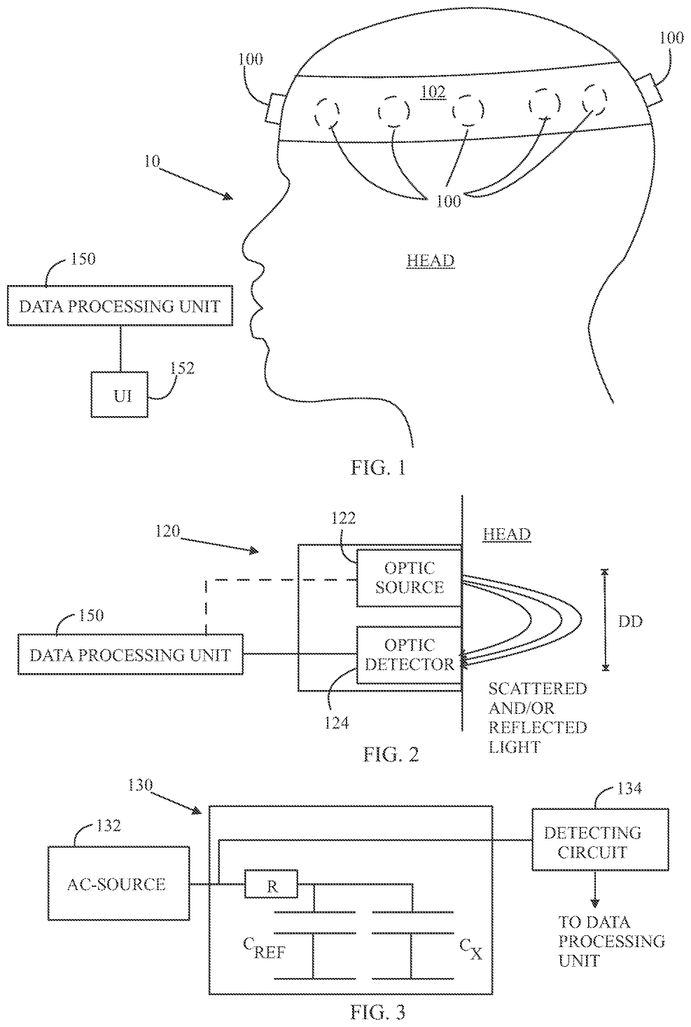

FIG. 1 illustrates an example of a measurement apparatus for measuring the intracranial dynamics, which comprises at least one sensing device 100. The apparatus for measuring the intracranial dynamics may comprise a support structure 102 to which the least one sensing device 100 is attached. The support structure 102 may be a band round the head or a cap on the head, for example, without limiting to these. Because the support structure 102 with the at least one sensing device 100 is easy to wear on the head, the apparatus is literally wearable. The at least one sensing device 100 transmits in a wired or wireless manner information of the intracranial dynamics to a data processing unit 150 which may be located at a distance from a mammal 10 such as a human being that has the at least one sensing device 100 attached to the head.

An example of the at least one sensing device 100 is an electrode of an electroencephalographic (EEG) electrode arrangement, which comprises the support 102 and at least two electrodes. The at least one sensing device 100 is in an electric contact with skin of a cranium of the mammal 10 an example of which is a human being. The sensing devices of the electroencephalographic electrode arrangement receive and sense direct-current (DC) electroencephalographic signals from the brain of the mammal 10. A person skilled in the art is familiar with the electroencephalographic electrode arrangement, per se, although the used bandwidth of the DC EEG is unusual, being in a range about 0 Hz to about 0.5 Hz.

Another example of the at least one sensing device 100 is an optic measurement arrangement 120, an example of which is illustrated in FIG. 2. The optic measurement arrangement 120 may comprise at least one optic radiation source 122 and at least one optic radiation detector 124. Each of the at least one optic radiation source 122 directs optic radiation toward the brain through the cranium, and the at least one optic detector 124 receives the optic radiation reflected and/or scattered therefrom. The at least one optic radiation source 122 may be in contact with the skin of the cranium. The at least one optic detector 124 may be in contact with the skin of the cranium. Alternatively, there may be a non-zero distance between the at least one optic radiation source 122 and the skin of the cranium. Correspondingly, there may be a non-zero distance between the at least one optic detector 122 and the skin of the cranium.

In an embodiment, the at least one optic radiation source 122 may be pigtailed such that the optic radiation is guided from the at least one optic source 122 to the skin of the cranium through an optic fiber. In an embodiment, the at least one optic detector 124 may be pigtailed such that the optic radiation is guided from the skin of the cranium through an optic fiber to the at least one optic detector 124. The optic fiber end (s) may be in contact with the skin of the cranium or be in a non-zero distance from the skin. The optic fibers are not separately illustrated in Figures.

Still another example of the at least one sensing device 100 is a capacitive sensor arrangement 130 where at least one sensing device which comprises at least two electrodes at separation distance of 2 cm to 5 cm from each other, for example, are attached on skin of a cranium of the mammal. An example of the capacitive sensor arrangement 130, illustrated in FIG. 3, may comprise a known resistance R and a parallel coupling of a known capacitance CREF and an unknown capacitance Cx in series, where the unknown capacitance Cx depends on permittivity of the head adjacent to the location where the electrode forming a part of the unknown capacitance Cx is. The permittivity, in turn, depends on a volume of the brain water and blood in the adjacency, i.e. the water within the cranium. An alternate current (AC) source 132, which may be a part of the sensing device 100 20 or a separate entity, may feed an electric signal to the resistance R and both of the known and unknown capacitances CREF and Cx, and a detecting circuit 134, which may be a part of the sensing device 100, a separate entity or a part of the data processing unit 150, may receive the electric signal affected by the RC-circuit of the resistance R and both of the known and unknown capacitances CREF and Cx. It is possible to solve the unknown capacitance Cx in a simple manner based on the waveform of the electric signal fed to the RC-circuit, the received electric signal, the resistance R and the known capacitance CREF, and the data processing unit 150 may provide the value of the unknown capacitance Cx in after its determination. In an embodiment, the input electric signal may include square waves and the frequency of the square waves may be in a kilohertz or megahertz range, for example.

The capacitive sensor arrangement is in proximity to or in physical contact with the skin of the cranium without having an electric contact to the skin of the cranium. The sensing devices of the capacitive sensor arrangement, which are electrodes, sense electric potential signals of the head.

In an embodiment, all the sensing devices 100 may be electroencephalographic electrodes. In an embodiment, all the sensing devices 100 may be optic detectors. In an embodiment, all the sensing devices 100 may be capacitive sensors. In an embodiment, the sensing devices 100 comprise a combination of at least two of different kinds of sensing devices 100. A combination may include at least one electroencephalographic sensing device 100 and at least one optic sensing device 100. Alternatively a combination may include at least one electroencephalographic sensing device 100 and at least one capacity sensing device 100. Still, alternatively a combination may include at least one optic sensing device 100 and at least one capacity sensing device 100.

The data processing arrangement 150 receives the electric signals from the at least one sensing device 100, and determines data on at least one of the following dynamics: glymphatic system, water within the cranium, brain tissue movements, water and/or electrolyte movements and intracranial pressure based on the electric signals. The water within the cranium includes the brain water, which may also be considered the CSF, and the blood. In an embodiment, the dynamics may be measured in a frequency band about 0 Hz to about 0.5 Hz. In an embodiment, the dynamics may be measured in a frequency band about 0 Hz to about 2.5 Hz. In an embodiment, the dynamics may be measured in a frequency band about 0 Hz to about 4 Hz. In an embodiment, the dynamics may be measured in a frequency band about 0 Hz to about 5 Hz. Dynamics of glymphatic system refers to the pulsatile activity related to its clearance function.

The data processing arrangement 150 computes values that represent relative volumes or concentrations of the water within the cranium. Correspondingly, the brain movement represents values of relative shrinkage or expansion of the brain within the cranium.

The measurement of the water within the cranium and brain water measurement are related to neurohydrodynamics. The brain water may also be considered to the same as or corresponding to cerebrospinal fluid (CSF), because from the measurement point of view those two are at least almost inseparable and they are within the cranium.

The data processing arrangement 150 outputs at least one piece of the data on the dynamics through a user interface 152.

That is, the determination of the dynamics may be based on only one of the following: the DC EEG measurement, the optic measurement or the capacitance measurement. Alternatively, the determination of the dynamics may be based on a combination of at least two of the following: the DC EEG measurement, the optic measurement or the capacitance measurement.

Dynamics in this document refers to motions that characterize a neurohydrodynamic and/or intracranial dynamics system, the motion being spatial variation of fluid(s) and/or tissue (s) as a function of time. Here the term “spatial” refers to one-, two- or three dimensional property such as length, area or volume. The water within the cranium may be measured as volume, the brain tissue movements may be measured as length, area or volume, and the intracranial pressure may be measured based on force per area (which is related to volume), for example.

It is a well-known fact that the absorption of light in brain tissue is governed by the Beer-Lambert law and the concentration of chromophores (such as unsaturated blood (Hb) i.e. deoxy-hemoglobin, saturated blood (HbO) i.e. oxy-hemoglobin, and water, which in this document is water within the cranium) can be calculated based on that. In this document, oxy-hemoglobin may be understood to mean oxidized hemoglobin of blood, and deoxy-hemoglobin may be understood to mean deoxidized hemoglobin of blood. The relative attenuation ao corresponds to relative loss of intensity of light. Attenuation a of optic radiation is caused by absorption and scattering. The measured intensity I of optic radiation can be expressed as follows:

I=I₀·e^{-[a·D]}=I₀·e^{-[(α}^{ₐ}^{+α}^{ₛ}^{)·D]}

where D is a length of propagation of optic radiation through medium, (αₐ+αₛ) denotes an attenuation coefficient a, αₐ denotes an absorption coefficient, αₛ denotes a scattering coefficient, e is the Napier number (about 2.718281828), I is intensity of the optic radiation passed through the medium i.e. the length D and I₀ is intensity of the optic radiation entering the medium. Relative attenuation a₀ refers to an attenuation that is measured when the optic radiation passes through a reference length D₀. Then the measured intensity I can be expressed in a mathematical for as:

I=I₀·e^{-[(a}^{₀}^{)·(D/D}^{₀}^{)]}=>I₀·e^{-[(α}^{ₐ₀}^{+α}^{ₛ₀}^{)·(D/D}^{₀}^{)]}

where αₐ₀ denotes a relative absorption coefficient and αₛ₀ denotes a relative scattering coefficient. The relative attenuation a₀ and the relative absorption coefficientαₐ₀ and the relative scattering coefficient αₛ₀ depend on the medium i.e. the brain tissue, the brain water and the blood. The relative attenuation ao and the relative absorption coefficient αₐ₀ and the relative scattering coefficient aso depend also on an oxygen level of the blood.

In an embodiment, the data processing arrangement 150 may determine, based on the electric signals, data on hydrodynamics of cerebral blood. The cerebral blood may also be called blood within the cranium.

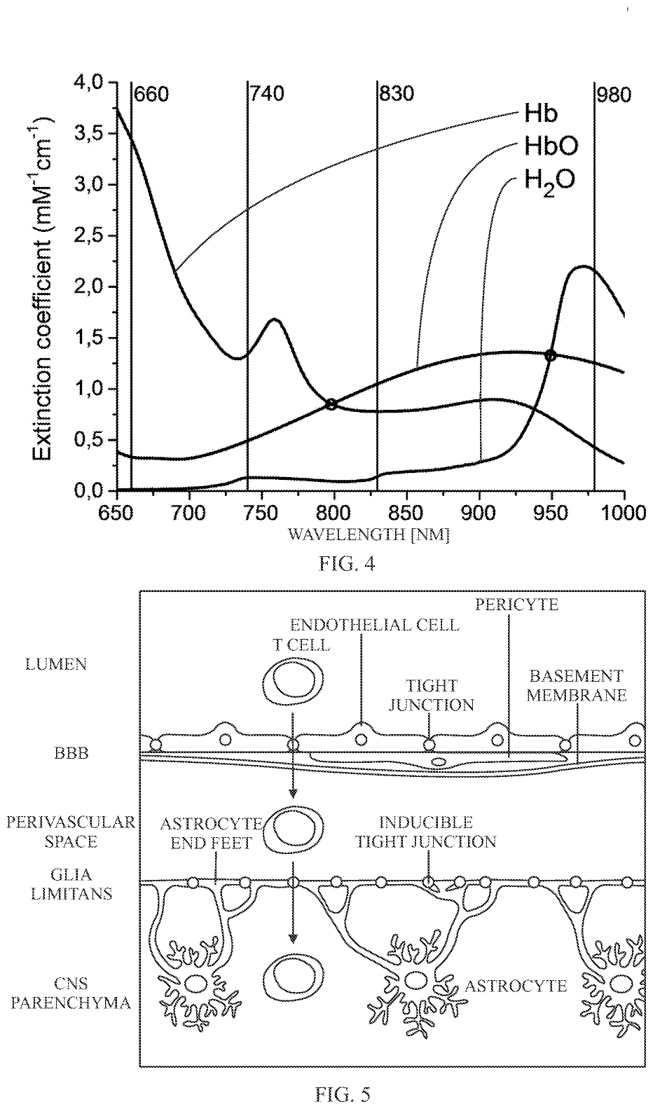

FIG. 4 illustrates absorption of water, saturated blood i.e. oxy-hemoglobin (HbO) and unsaturated blood i.e. deoxy-hemolobin (Hb) in a spectral band for water and hemoglobin measurements. This is the so-called optic brain window spectral range where photons can reach the cerebral cortex in the head of the mammal such as a human being. In this document, the spectral range that is used for the measurement is not limited to those shown or referred to, but also longer wavelengths may be used (for example, so-called second brain window).

FIG. 4 shows that for unsaturated blood i.e. deoxy-hemolobin Hb and saturated blood i.e. oxy-hemolobin HbO, there is an isosbestic point located approximately at about 800 nm and for the saturated blood i.e. oxy-hemolobin HbO and water at approximately 950 nm. As an example, measurement wavelengths at 660 nm, 740 nm, 830 nm and 980 nm are marked in FIG. 4 for calculating concentrations for unsaturated blood i.e. oxy-hemolobin HbO, saturated blood i.e. deoxy-hemolobin Hb and the water H₂O within the cranium simultaneously or alternatively. However, also other combination of wavelengths may be used. But, at least one wavelength may be below about 800 nm, one between about 800 nm and about 940 nm for calculating unsaturated blood i.e. deoxy-hemolobin Hb and saturated blood i.e. oxy-hemolobin HbO. At least one wavelength may be one above 940 nm for calculating water within the cranium in addition to the measurement of the unsaturated blood i.e. deoxy-hemolobin Hb and the saturated blood i.e. oxy-hemolobin HbO.

Calculated concentration values for deoxy-hemoglobin Hb, oxy-hemoglobin HbO and water within the cranium comprise the total measurement volume of the optode. In an embodiment, the optic source 122 and the optic detector 124 may be spaced at a non-zero distance DD such as about 3 cm to about 4 cm from each other, for example. In an embodiment, it is possible to measure the effect of dynamics caused by the skin layer when having an additional detector at short distance (usually less than 1 cm).

All values may be calculated separately, and the effect of skin may be subtracted from the values measured at a longer source-detector distance to improve the detection of signal dynamics in deeper layers. In an embodiment, the optic source 122 and the corresponding optic detector 124 are optically coaxial. Alternatively or additionally, by using commonly known time of flight technique, source and detector may be placed close to each other, where the measurement depth is determined by the photons' time of flight (arrival time in the detector). Or, different frequency modulation techniques can be used to determine the measurement depth based on phase shift.

In an embodiment (see FIG. 4), the optic measurement arrangement (120) is configured to measure the brain tissue at at least one optic wavelength band, which is dominantly attenuated by the water within the cranium, and at least one optic wavelength band, that is dominantly attenuated by the blood within the cranium in order to separately measure the dynamics of the water within the cranium and the cerebral blood where dynamical changes between the blood and water within the cranium reflect glymphatic activity and brain tissue pulsations.

In a similar manner to the measurement of water within the cranium, the data processing arrangement 150 may compute values that represent relative volumes or concentrations of the blood within the cranium.

In an embodiment (see FIG. 4), the optic measurement arrangement 120 may measure the brain water at at least one wavelength dominantly absorbed by water and the cerebral blood based on at least one of the following: at least one wavelength dominantly absorbed by deoxy-hemoglobin and/or at least one wavelength dominantly absorbed by oxy-hemoglobin. Here, the term deoxy-hemoglobin refers to blood that is unsaturated with oxygen and the term oxy-hemoglobin refers to blood that is saturated with oxygen. The deoxy-hemoglobin may reside in veins whereas oxy-hemoglobin may reside in arteries.

In an embodiment (see FIG. 4), the data processing arrangement 150 may determine, based on the electric signal(s) from the optic measurement arrangement 120, data on dynamics of the oxy-hemoglobin of the cerebral blood.

In an embodiment (see FIG. 4), the data processing arrangement 150 may determine, based on the electric signal(s) from the optic measurement arrangement 120, data on dynamics of the deoxy-hemoglobin of the cerebral blood.

In an embodiment (see FIG. 4), the data processing arrangement 150 may determine, based on the electric signals from the optic measurement arrangement 120, data on dynamics of total hemoglobin that is of a sum of deoxy-hemoglobin and oxy-hemoglobin measurements.

In an embodiment (see FIG. 4), the optic measurement arrangement 120 may measure the water within the cranium at a wavelength band including the wavelength of 980 nm and the cerebral blood at least one of the following: a wavelength band including the wavelength of 660 nm and/or 690 nm, a wavelength band including the wavelength of 740 nm, a wavelength band including the wavelength of 830 nm. That is, two of the wavelengths 660 nm, 740 nm or 830 nm may be used, for example. In an embodiment, the wavelength 660 nm may be replace with 690 nm, for example.

In an embodiment (see FIG. 4), the optic measurement arrangement 120 may measure the water within the cranium at a first wavelength band from 940 nm to 1100 nm, and the cerebral blood at least one of the following: a second wavelength band from 600 nm to 800 nm and a third wavelength band from 800 nm to 940 nm. The optic measurement arrangement 120 performs the measurement such that the first and second wavelength bands are separated by at least about 20 nm. In a similar manner, the second and the third wavelength bands are separated by at least about 20 nm.

In an embodiment (see FIG. 4), the optic measurement arrangement 120 may measure the cerebral venous blood i.e. the deoxy-hemoglobin at the wavelength band including a wavelength below 800 nm and the cerebral arterial blood i.e. the oxy-hemoglobin at the wavelength band above 800 nm.

In an embodiment, the data processing unit 150 may detect an opening of a blood-brain-barrier (BBB) based on at least one of the hydrodynamics of the cerebral arterial blood and the hydrodynamics of the cerebral venous blood.

In an embodiment, the data processing unit 150 may detect changes in regional concentration changes of the oxy-hemoglobin and deoxy-hemoglobin and water within the cranium following blood opening of the BBB.

In an embodiment, the data processing unit 150 is configured to detect an opening of the BBB based on dynamics of water within the cranium.

FIG. 5 illustrates a neurovascular unit: The BBB comprises three layers 1) endothelium woven together with tight junctions, 2) basement membrane collagen mesh, and 3) pericyte/astrocyte lining (glia limitans) that is somewhat separated from the layers 1 and 2 by the glymphatic paravascular spaces.

The CSF is produced by the choroid plexuses located in the ventricles. It flows from the lateral ventricles to the third ventricle and then to the fourth ventricle and exits to the subarachnoid space. From the subarachnoid space, it flows to the arachnoid granulations and then enters the venous circulation from the superior sagittal sinus.

The three layer i.e. the blood-brain barrier, BBB, (i.e. endothelium, basement membrane and astrocyte/pericyte) between blood and the brain tissue that maintains the homeostasis of the electrolyte gradient in the brain. The BBB does not penetrate water soluble polar molecules and normally has a potential of mV between blood and brain tissue interstitium. For historical and semi-conductor hardware reasons, the brain electrophysiological signal from electroencephalogram (EEG), does not usually measure the 5 mV DC-potential across the BBB, as its origin has been considered non-neuronal and therefore less interesting. The DC-EEG potential is >1000× greater than the more commonly measured EEG (alpha, beta, gamma, delta, or theta) rhythms induced by neurons and still this valuable information is usually filtered out by 0.5 Hz high-pass filtering.

This document describes how to utilize this possibility to quantify very low frequency (<0.1 Hz, at least approximately) vasomotor fluctuations of blood vessel wall smooth muscle with DC-EEG potential shifts and the DC-EEG has been also shown to reflect changes in the permeability of human BBB. We can use the same DC-EEG techniques to quantify VLF potentials in sleep, when the glymphatic water trafficking over the BBB glia limitans increases. Brain diseases affecting the blood vessel wall (Alzheimer, stroke, trauma, epilepsy, etc.) can affect the DC-EEG potentials and its variation directly also. For instance in epilepsy the respiratory 0.2 Hz brain pulsation has been shown to be driving classical faster EEG rhythmic activity and this driving changes in sleep as well. Classical The NIRS can monitor the power of three glymphatic drivers (vasomotor, cardiovascular and respiratory pulsations) and the power changes can be analysed with respect to DC-EEG permeability changes to identify how much each pulsation drives the permeability.

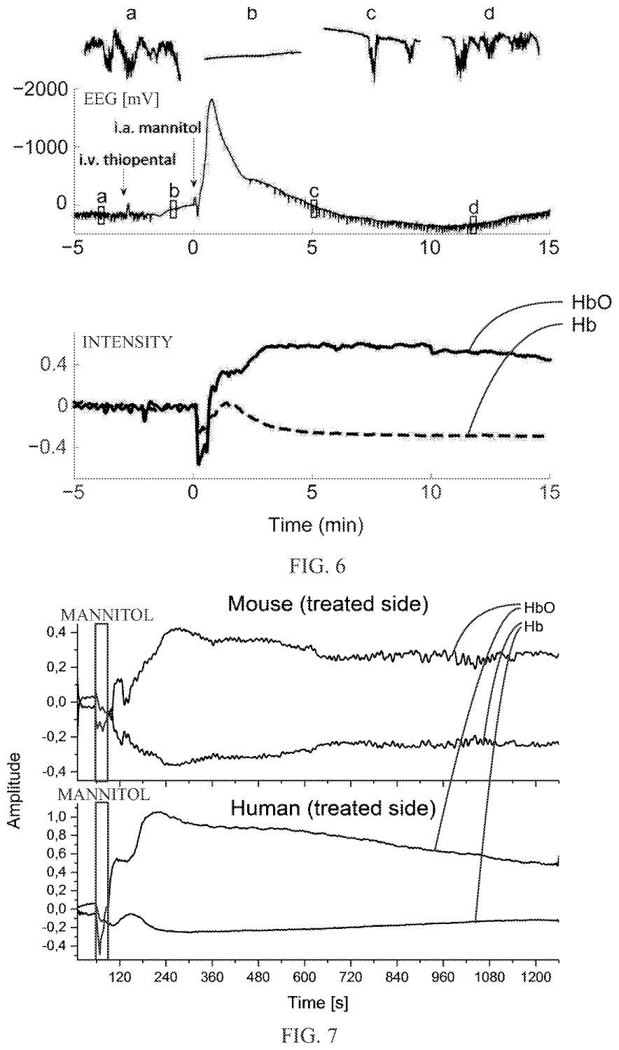

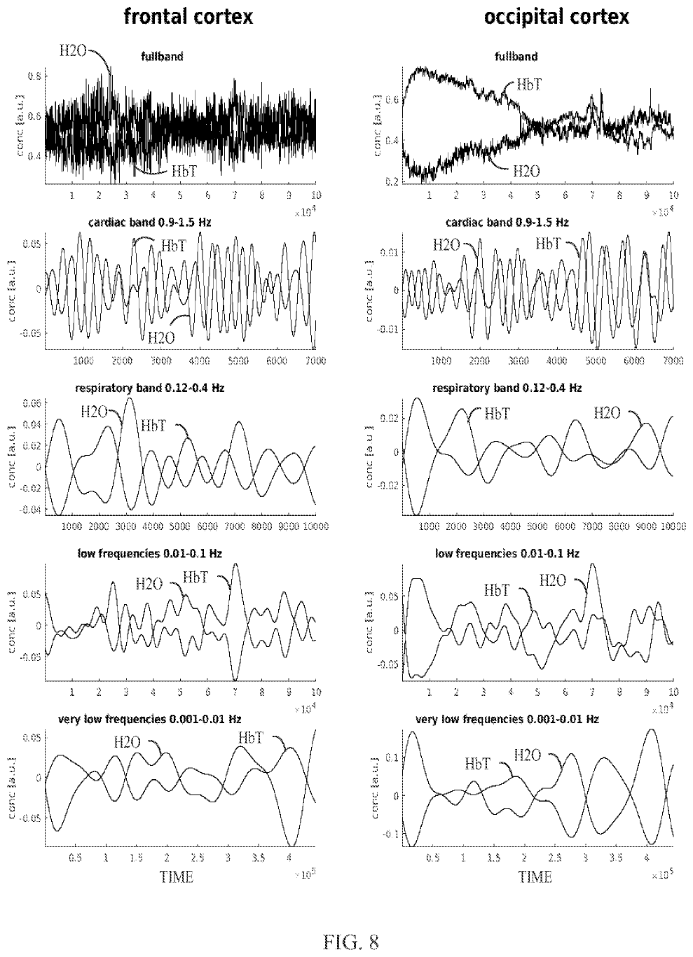

The DC-EEG may be used to measure the potential of a blood-brain barrier when the about 0.5 Hz highpass is not used and the data is measured with digital amplifiers that have a wide sensitivity range. FIG. 6 illustrates an example that the potential of DC-EEG is unequivocally due to the osmotic opening of the BBB in a human subject when neuronal function is absent due to deep thiopental anesthesia prior to i.a. mannitol based opening of the BBB during treatment of primary central nervous system lymphoma. At the same time, strong changes in blood flow dynamics are also seen in the optic measurement. The a-d signals represent zoomed time periods also representatively marked in the DC-EEG signal below. During the time of the mannitol induced BBB opening, where in b) the neuronal activity has been momentarily silenced with i.v. thiopental anesthesia bolus given just before mannitol in the PCNSL (primary central nervous system lymphoma) treatment protocol. Notice the return of the neural activity around d). To determine BBB opening level may be of interest in brain drug delivery used in brain cancer chemotherapies, for example.

FIG. 6 illustrates examples of time series showing deoxy-hemoglobin Hb, oxy-hemoglobin HbO and EEG signals measured simultaneously when measured during mannitol infusion/BBB opening. The phase and amplitude differences between the deoxy-hemoglobin Hb and oxy-hemoglogin HbO and EEG can be used to diagnose the glymphatic activity and BBB permeability. Importantly, direct water quantitation with NIRS reflect whether the DC-EEG permeability changes are related to water dynamics and how much this drive is present in individual sleep or disease (s).

In an embodiment an example of which is illustrated in FIG. 6, the data processing unit 150 may determine permeability changes including opening of the BBB based on a synchronous amplitude change of both a signal of the optic measurement arrangement 120 and a direct-current (DC) electroencephalographic signal of the electroencephalographic electrode arrangement of the at least one sensing device 100.

In an embodiment an example, the data processing unit 150 may determine permeability changes including opening of the BBB based on a synchronous amplitude change of both a signal of the optic measurement arrangement 120 and a signal of the capacitive measurement arrangement 130. Furthermore, the water sensitive near-infrared spectroscopy probe may determine the alternation in brain water content following the BBB permeability changes following therapeutic procedures and pathological incidents.

In an embodiment an example, the data processing unit 150 may determine permeability changes including opening of the BBB based on a synchronous amplitude change of both a direct-current (DC) electroencephalographic signal of the electroencephalographic electrode arrangement of the at least one sensing device 100 and a signal of the capacitive measurement arrangement 130.

In an embodiment, the data processing unit 150 may determine permeability changes including opening of the BBB based on a signal of the capacitive measurement arrangement 130.

In an embodiment, the data processing unit 150 may determine opening of BBB based on a signal of a direct-current (DC) electroencephalographic signal of the electroencephalographic electrode arrangement of the at least one sensing device 100.

In an embodiment, the data processing unit 150 may determine opening of BBB based on a signal of a direct-current (DC) electroencephalographic signal of a signal of the optic measurement arrangement 120.

The momentary increase of the permeability over the BBB (and glia limitans) is shown in FIGS. 6 and 7 after i.a. mannitol injections. The DC-EEG reflects therefore the permeability over the blood-brain barrier and the glymphatic activity which the data processing unit 150 may detect and determine. In normal physiological condition, it is the BBB layers 1 and 2 that remain intact but in conditions like sleep and after disease infiltrations of the perivascular space, the glia limitans may become more permeable and this may be monitored externally with combined measurements of water within the cranium and DC-EEG as slow modulations of the

BBB potential.

FIG. 7 illustrates the dynamics of the cerebral arterial blood i.e. oxy-hemoglobin HbO and the cerebral venous blood i.e. deoxy-hemoglobin Hb at both sides of the moment of opening of the BBB. In FIG. 7, the opening of the BBB is caused by infusion of mannitol.

In an embodiment which is supported by examples of FIGS. 6 and 7, the data processing unit 150 may determine a glymphatic activity based on at least one of the hydrodynamics of the cerebral arterial blood and the cerebral venous blood.

FIGS. 6 and 7 illustrate examples of time series showing deoxy-hemoglobin Hb and oxy-hemoglobin HbO signals measured simultaneously. FIG. 7 illustrate an example of time series showing deoxy-hemoglobin Hb and oxy-hemoglobin HbO signals measured simultaneously during internal carotid artery mannitol infusion dufing the BBB opening in both mouse and human. The phase and amplitude differences between deoxy-hemoglobin Hb and oxy-hemoglobin HbO may be used to detect regional blood flow during the BBB permeability increase.

In normal physiological (non-anesthetized, without mannitol) states like sleep the DC-EEG pulsations are not as remarkable, but more subtle, yet they have been shown to increase in sleep. During this increase in oscillations, also the DC-EEG drive on faster rhythms changes. This causative drive changes forms another quantifiable metric of the BBB state in addition to the very low frequency power of the DC-potential. A third indicator is the relationship between NIRS (the optic measurement) and DC-EEG pulsations, phase, amplitude and directed phase entropy being measured quantities.

In an embodiment, the data processing unit 150 may form data on glymphatic water as a difference between the dynamics of water and the blood within the cranium. The data may based on volumes of the water and blood, for example.

The data processing unit 150 may form an instantaneous volume together with the dynamics of the volume. In this example, blood-bound water may be subtracted from water within cranial: GBW=H2O−HbT, where H2O is water within the cranium, GBW is glymphatic brain water, HbT is total blood response, which is a sum of deoxy-hemoglobin Hb and oxy-hemoglobin HbO (HbT=Hb+HbO). The remaining dynamics in glymphatic brain water signal is caused for the most part by CSF, which reflects dynamics of glymphatic circulation accurately.

Although the water value may include both intracellular and extracellular compartments of water, the intracellular part is not expected to display great fluctuations in water concentration over a short period of time (such as 10 minutes).

Furthermore, as no significant dynamics occur in water in the skull or skin layer, it can also be concluded that the water dynamics in the signals are mainly caused by the physiological effects occurring below the skull layer, particularly in the CSF layer and in the subarachnoid space. Based on simulations, the dynamics of water may be sensed by approximately 20% of photons (when using wavelength near 980 nm), which travel along and through the CSF into the cortex, thus by using such wavelength alone it can be possible to sense the brain water and CSF activity.

Time series showing water within the cranium H2O and oxy-hemoglobin HbO signals measured simultaneously are shown in FIG. 8.

In an embodiment an example of which is illustrated in FIG. 8, the data processing unit 150 may determine the glymphatic activity based on inverse correlation, phase difference, and/or entropy transfer of the data of the water within the cranium and the cerebral arterial blood. The data may be measured using the optic measurement arrangement 120.

In the example of FIG. 8, the signals oscillate in almost opposite phases. The phase differences and amplitude differences may be utilized to detect the glymphatic activity. Furthermore the variance in the cardiac HbO pulsations has been shown to be increased in amyloid deposit that prevent the glymphatic water dynamics in Alzheimer's disease (AD). The variance of the HbO cardiac pulsations can be used as a marker for AD then. Similarly, the phase and amplitude differences between water within the cranium H2O and deoxy-hemoglobin Hb as well as water within the cranium H2O and total blood response HbT may be used to detect the glymphatic activity. This can quantify the amount of water being transferred with respect to the three glymphatic pulsations. For instance, the pulsations of HbO and Hb may be strong but water does not pulsate, indicates a failing glymphatic circulation.

In an embodiment which is supported by FIG. 9, the data processing unit 150 may determine activity of water based on an envelope of a signal of the data of water within the cranium during brain activation. The data processing unit 150 may determine the glymphatic activity based on an envelope of a signal of the data on water within the cranium. The functional MRI MREG BOLD (blood oxygen level dependent) responses during repetitive 15 sec visual stimuli drop during the 2^{nd} stimulus and during this drop, the water content of the brain increases. In other words the blood oxygenation level (HbO) and its responsive increase during activation decrease in the presence of water increase in the cortex. This is also observed in the water/HbO/Hb measurements in FIG. 8 as opposite pulsations in water content with regards to blood volume. This could mean that during a vasomotor tone increase the brain CSF/interstitial water content increases. And that vasomotor functions as a pump to increase local water content. This inverse correlation may be altered by disease or sleep in a detectable way and may relate to wellbeing of the mammal, which may be relieved by changing one or more habits, or even a more serious health issue.

FIG. 9 illustrates time series during visual tasks (marked with arrows pointing to the vertical rectangles) showing the upper envelopes of water (free) within the cranium, and magnetic resonance encephalography (MREG) time series, when measured simultaneously. The shown envelopes oscillate in opposite phases. These phase differences can be used to detect the glymphatic clearance and water transfer activity. This is an important, independent measure showing the same phenomenon as that shown in FIG. 8. The inverse variation i.e. the dynamics of the water within the cranium reflects the glymphatic activity and clearance.

In an embodiment, the data processing unit 150 may determine data on the glymphatic activity based on the data on the hydrodynamics of water within the cranium and the physiological model. Dynamics in the calculated signal of water within the cranium are assumed to be caused by: (1) volume changes of the brain water, and (2) volume changes in blood-bound water. Calculated data on the water within the cranium reflects the glymphatic circulation.

In an embodiment, the data processing unit 150 may detect the opening of the BBB based on electric signals from the electroencephalographic electrode arrangement of the at least one sensing device 100.

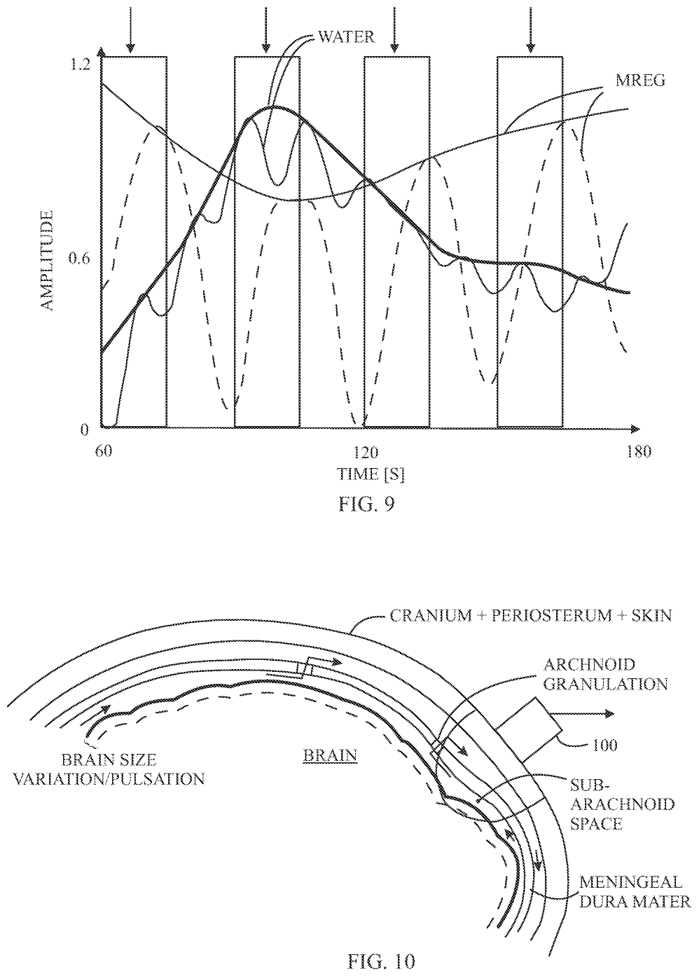

In an embodiment, the data processing unit 150 may determine the intracranial pressure and/or stiffness of the brain based on a waveform of a signal of the data on the hydrodynamics of at least one of the brain water and the cerebral blood. In an embodiment, the data processing unit 150 may determine the intracranial pressure and/or stiffness of the brain blood vessels based on a waveform of a signal of the data on the hydrodynamics of the cerebral blood only. The waveform is caused by head movement, heartbeat and/or breathing. The movement of the brain is illustrated in FIG. 10. The waveform may be caused by the heartbeat. The waveform may have a shape of a pulse, for example.

In an embodiment which is illustrated in FIG. 10, the at least one sensing device 100 such as the electroencephalographic electrode arrangement, the optic measurement arrangement 120 and/or the capacitive measuring arrangement 130 may be located at or close to a subarachnoid space. The at least one sensing device 100 may be located at the surface of the head in occipital, temporal, perietal and/or forehead, for example. Additionally or alternatively, the at least one sensing device 100 may be located in any other location.

In an embodiment, the data processing unit 150 is configured to form correlation relating to a phase difference and/or amplitude difference at specific frequencies of very low frequencies (0.001 Hz-0.01 Hz), low frequency (0.01 Hz-0.1 Hz), respiratory band and cardiac band, and their modulatory interconnections between dynamics of blood and water within the cranium to determine data on glymphatic activity and/or BBB.

The dynamics of oxy-hemoglobin signal and glymphatic signal of the water within the cranium measured in subarachnoid space reflect the dynamics of the glymphatic clearance. FIG. 10 also illustrates an example of the measured volume (marked with hemisphere under the sensing device 100), which makes it possible to detect the glymphatic clearance. However, the placement of the sensing device 100 is not limited to this position. In an embodiment, there are a plurality of the sensing devices 100 around the head, for example. The measurement may be performed also in the frontal part of the head, for example, without limiting to this location.

Overall brain stiffness may be considered as a combination of the biomechanical properties of the pia-arachnoid, grey matter, white matter, cerebral blood vessels, blood, brainwater/cerebrospinal fluid (CSF), and brainwater/cerebrospinal fluid (CSF) pressure. All of these components may affect the brain stiffness, and the stiffness is dependent on age as found after comparing temporal and parietal cerebral lobes, subcortical gray matter structure, the caudate and the putamen of the healthy adult brain to the adolescents. Accoding to the prior art, the brain stiffness and the intracranial pressure (ICP) may be measured in-vivo only invasively using indentation probe.

By measuring the optic radiation signal scattered and/or reflected from the brain cortex and analysing its cardiac/volumetric pulse shape, reflecting also pulsational movement of the brain, the brain stiffness may be estimated. The dashed-line and the thick line of the outline of the brain in FIG. 10 illustrate the amplitude of the brain pulsational movement of the brain between shrinkage and expansion. If stiffness increases, glymphtic clearance stops and disorders such as epilepsy, cognitive decline and/or Alzheimers disease may develop, for example.

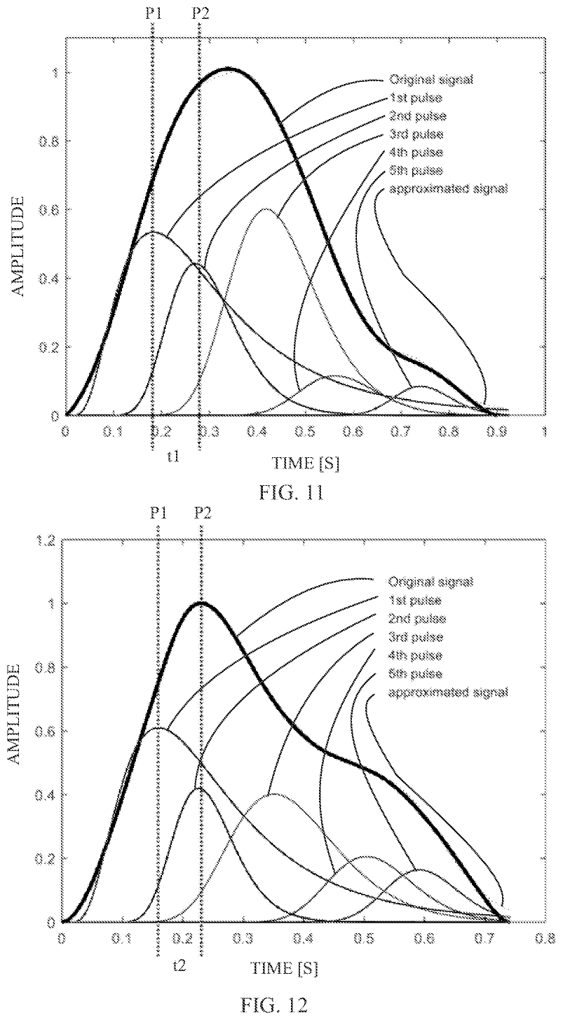

When the brain is elastic or plastic, the cardiac pulsation in the received optic and/or electric signal from the head is also affected, particularly in the trailing edge. In that case, the situation may be interpreted such that the blood vessels succesfully compensate the pulse pressure. Thus, this pulse pressure travels slowlier, and its reflected wave and/or the peak of amplitude appears later and its trailing edge is longer. Such a situation is illustrated in FIG. 11, which shows a waveform of a young person.

When the brain becomes stiff or rigid, the cardiac pulsation in the received optic signal from the head is also affected, particularly the first reflected wave at the rising edge. In that case, the situation may be interpreted such that the blood vessels fail to compensate the pulse pressure. Thus, this pulse pressure travels fast, and its reflected wave and/or the peak of amplitude appears soon sooner than in the case of the elastic brain. Such a situation is illustrated in FIG. 12, which shows a waveform of a elder person.

It is apparent that the distance between the points P1 and P2 of the elastic brain is longer than the one of the stiffer brain, i.e t1 from point P1 to point P2 is longer than t2. Similarly, differences between pulse shapes of a healthy patient and an Alzheimer's disease patient of the same age may be detected.

In an embodiment, the data processing unit 150 may compare the waveform of the pulse with a reference waveform, which is based on a physiological model of a head, for determining at least one of the following: brain stiffness, the intracranial pressure and deviation from the physiological model. In an embodiment, the physiological model of the head may be that of a healthy person. In an embodiment, the physiological model of the head may be that of a person of a certain age. In an embodiment, the physiological model of the head may be that of a man or a woman. In an embodiment, the physiological model of the head may be that of a person with a known issue with the neurohydrodymanics such as normal pressure hydrocephalus (NPh), neurodegeneration, dementia, Alzheimer's disease (AD), Chiari, increased intracranial pressure, stroke, trauma, tumor or the like. In an embodiment, the data processing unit 150 may have a data bank of different physiological models of the head or the data processing unit 150 may have access to the of different physiological models of the head for comparison between the measurement and the models of the data bank.

Various calculation techniques can be used to analyze the pulse shape and its dependence on the elasticity of the brain. In an embodiment examples of which are illustrated in FIGS. 11 and 12, the data processing unit 150 may decompose the waveform into decompositions 1^{st} pulse, 2^{nd} pulse, 3^{rd} pulse, 4^{th} pulse and 5^{th} pulse, and determine at least one of the following: brain stiffness, the intracranial pressure and deviation from the physiological model based on the decompositions. The number of the decomposition is 2 or larger.

The original pulse pressure and its first reflection may be captured by the pulse decomposition analysis and the peak time may be used further to evaluate accuracy of the brain stiffness.

The decompositions 1^{st} pulse, 2^{nd} pulse, 3^{rd} pulse, 4^{th} pulse and 5^{th} pulse have different heights and widths in FIGS. 11 and 12. The pulses 4^{th} pulse and 5^{th} pulse at the trailing edge are lower in a stiff brain than in an elastic brain, for example. Because there is a wide variety of possibilities how the waveform relates to the brain stiffness it is possible, in an embodiment, that the recognition of the brain stiffness from the waveform is based on artificial intelligence, neural network and/or machine learning methods, for example. Alternatively or additionally, the brain stiffness may be determined based on image processing of a curve of the waveform.

In an embodiment, the waveform may be decomposed based on an integral transform. In an embodiment the integral transform may be the Fourier transform, the Laplace transform, which the person skilled in the art is familiar with.

In the Gaussian transform, 3 to 5 individual log-normal waves may be extracted, for example (there are 5 log-normal waves in FIGS. 11 and 12). The Gaussian transform may be expressed mathematically as

$$f ( A , c , σ ) = {\sum }_{n = 1}^{N} \frac{{A}_{n}}{x {σ}_{n}} {e}^{\frac{- {( ln ( \frac{x}{{c}_{n}} ) )}^{2}}{2 {σ}_{n}^{2}}}$$

where N is the number of the decompositions, n is the index of the summation, A is amplitude, c is a center point and σ is the standard deviation. As a result, each optically measured waveform may be represented by fifteen parameters when using 5 decompositions, the values of parameters being estimated by a nonlinear curve fitting with constraints, for example.

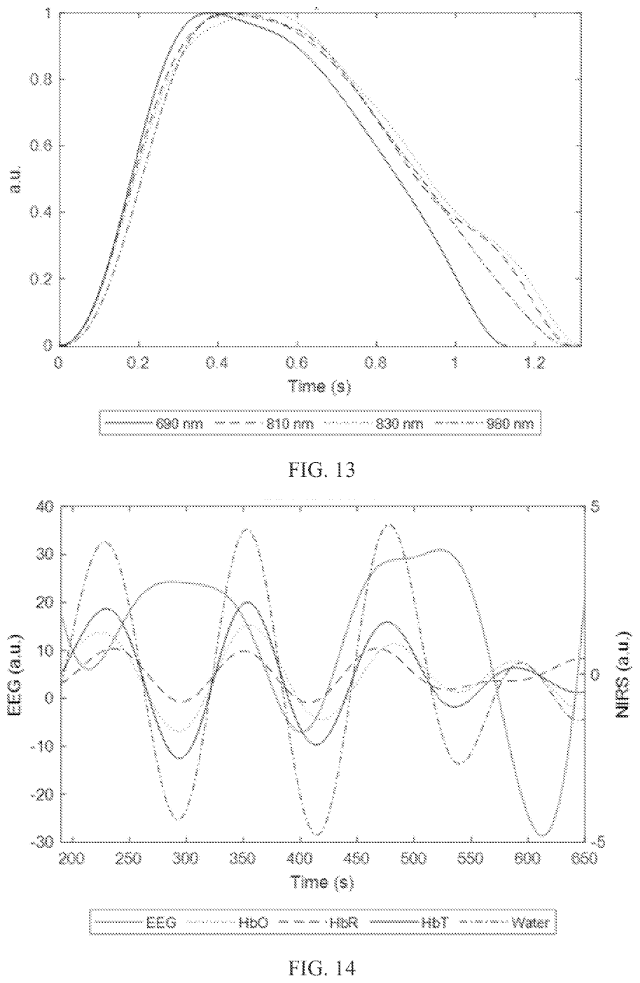

FIGS. 13 to 15 illustrate examples relating to what is explained earlier in this document. FIG. 13 illustrates an example of variation in recorded brain pulse waveforms at different wavelengths used in the analysis method.

FIG. 14 illustrates an example of recorded signals in time domain used in the analysis method. The delays, amplitude changes and other variations in fluctuations between these signals may be seen as points of interest.

FIG. 15 illustrates an example of the spectral powers the signals recorded from the brain and used in the analysis method. The relations between frequency components of the selected signals, particularly at typical frequency ranges for breathing and cardiac activity, as well as at very low frequency range may be seen as points of interest.

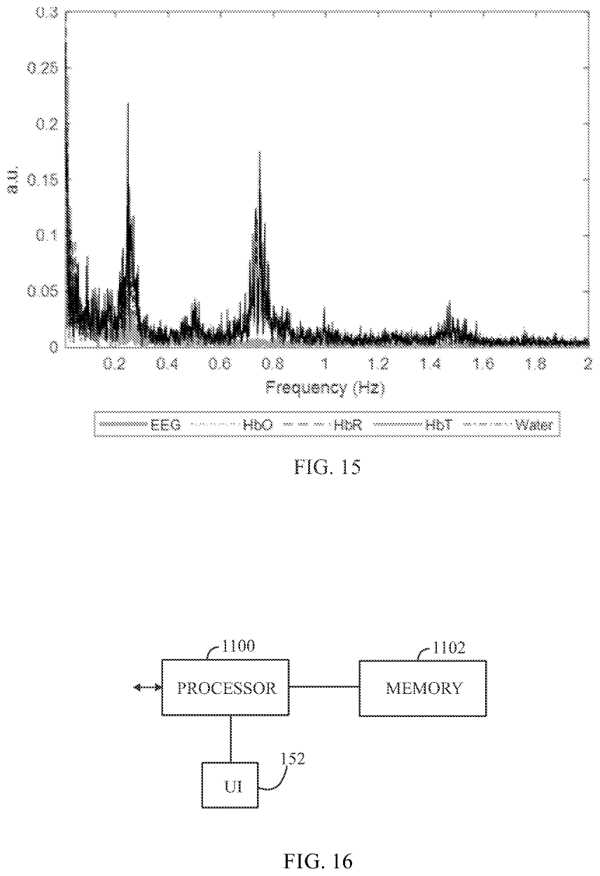

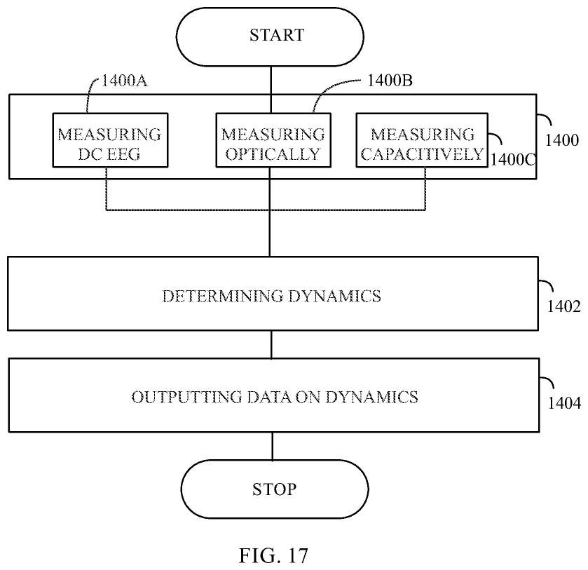

In an embodiment an example of which is illustrated in FIG. 16, the data processing unit 150 may comprise for measuring the intracranial dynamics one or more processors 1100, one or more memories 1102 including computer program code. The one or more memories 1102 and the computer program code may, with the one or more processors 1100, cause the data processing unit 150 for measuring the intracranial dynamics at least to receive the electric signals from the at least one sensing device 100, determine dynamics of the at least one of the following: water within the cranium, the brain water, the brain tissue movements and the intracranial pressure based on the electric signals, and output the at least one piece of the data on the dynamics through the user interface 152.

FIG. 17 is a flow chart of the measurement method of the intracranial dynamics. In step 1400, at least one of the following is performed:
in step 1400A measuring direct-current (DC) electroencephalographic signals from the brain with an electroencephalographic electrode arrangement in electric contact with skin of a cranium,
in step 1400B directing optic radiation toward the brain, and receiving the optic radiation reflected and/or scattered therefrom with an optic measurement arrangement 120, and
in step 1400C measuring electric potential signals of the brain with a capacitive sensor arrangement 130 in proximity without having an electric contact to the skin of the cranium.

In step 1402, at least one of the following dynamics is determined based on electric signals from the at least one sensing device 100 by the data processing arrangement 150: water within cranium, brain tissue movements, water and/or electrolyte movements and intracranial pressure based on the electric signals.

In step 1404 the at least one piece of the data on the dynamics is output through a user interface 152.

The method shown in FIG. 17 may be implemented as a logic circuit solution or computer program. The computer program may be placed on a computer program distribution means for the distribution thereof. The computer program distribution means is readable by a data processing device, and it encodes the computer program commands and carries out the measurements.

The apparatus for measuring the intracranial dynamics monitors glymphatic activity in a non-invasive manner by measuring simultaneously brain's hydro-, hemo- and electrophysiological dynamics. In particular, it can measure optically 1) contents of the water within the cranium. Interaction of the water with cerebral blood volume and hemodynamics can also be measured. It is also possible to measure 2) brain tissue/blood volume/water pulsation related measures reflecting brain stiffness and intracranial pressure (ICP). 3) DC-EEG potential measurement may be performed between blood and brain tissue reflecting permeability changes in glia limitans interface of the blood brain barrier (BBB) with the glymphatic paravascular spaces.

Combination of these parameters provide an estimate for the glymphatic water and metabolite exchange across the brain.

The wearable apparatus for measuring intracranial dynamics and glymphatic functions that is described in this document enables early detection of pathological changes preceding diseases such as Alzheimers disease, improves possibility to improve the wellbeing and possibly also diagnostics of brain diseases and helps in search for development of drugs/treatments for brain diseases. Easy to wear tools (band or cap) enable also advanced analysis of upright position on water within the cranium and hydrostatic pressure effects of the brain water, as recent research also indicates that the spinal canal may be an important part of the glymphatic clearance. Such monitors should also develop markets, increase financial and industrial entreprises and bring work to people. Ultimately what is presented in this document has the potential to increase human life quality and even longevity.

The computer program may be distributed using a distribution medium which may be any medium readable by the controller. The medium may be a program storage medium, a memory, a software distribution package, or a compressed software package. In some cases, the distribution may be performed using at least one of the following: a near field communication signal, a short distance signal, and a telecommunications signal.

It will be obvious to a person skilled in the art that, as technology advances, the inventive concept can be implemented in various ways. The invention and its embodiments are not limited to the example embodiments described above but may vary within the scope of the claims.

1. An apparatus for measuring intracranial dynamics, wherein the apparatus comprises at least one sensing device of the following:
an electroencephalographic electrode arrangement, which is configured to be in electric contact with skin of a cranium, and sense direct-current electroencephalographic signals from the brain,
an optic measurement arrangement configured to direct optic radiation toward the brain through the cranium, and receive the optic radiation reflected and/or scattered therefrom, and
a capacitive sensor arrangement configured to be in proximity without having an electric contact to the skin of the cranium, and sense electric potential signals of the head, and the apparatus additionally comprises
a data processing arrangement configured to receive electric signals from the at least one sensing device, and determine data on at least one of the following dynamics: glymphatic system, water within the cranium, brain tissue movements, water and/or electrolyte movements and intracranial pressure based on said electric signals from the at least one sensing device; and
the data processing arrangement is configured to output of at least one piece of the data on the dynamics through a user interface.

2. The apparatus of claim 1, wherein the optic measurement arrangement is configured to measure the brain tissue at at least one optic wavelength band, which is dominantly attenuated by the water within the cranium, and at least one optic wavelength band, which is dominantly attenuated by the blood within the cranium in order to separately measure the dynamics of the water within the cranium and the cerebral blood where dynamical changes between the blood and water within the cranium reflect glymphatic activity and brain tissue pulsations.

3. The apparatus of claim 1 wherein the data processing unit is configured to detect an opening of a blood-brain-barrier based on concentration changes of the oxy-hemoglobin, deoxy-hemoglobin and water within the cranium.

4. The apparatus of claim 1 wherein the data processing unit is configured to form data on glymphatic activity as a difference between the dynamics of water and the blood within the cranium.

5. The apparatus of claim 4, wherein the data processing unit is configured to determine the glymphatic activity based on correlation of the data on the water within the cranium and the cerebral arterial blood.

6. The apparatus of claim 4, wherein the data processing unit is configured to determine the glymphatic activity based on an envelope of a signal of the data on water within the cranium.

7. The apparatus of claim 1, wherein the data processing unit is configured to determine the intracranial pressure and/or stiffness of the brain based on a waveform of a signal of the data on the hydrodynamics of at least one of the following: the water within the cranium and the cerebral blood, the waveform being caused by head movements, heartbeat and/or breathing.

8. The apparatus of claim 7, wherein the data processing unit is configured to compare the waveform with a reference waveform, which is based on a physiological model of a brain, for determining at least one of the following: brain stiffness, the intracranial pressure and deviation from the physiological model.

9. The apparatus of claim 7, wherein the data processing unit is configured to decompose the waveform into decompositions, and determine at least one of the following: brain stiffness, the intracranial pressure and deviation from the physiological model.

10. The apparatus of any preceding claim, wherein data processing unit is configured to form correlation relating to a phase difference and/or amplitude difference at specific frequencies of very low frequencies (0.001 Hz-0.01 Hz), low frequency (0.01 Hz-0.1 Hz), respiratory band and cardiac band between dynamics of blood and water within the cranium to determine data on glymphatic activity and/or BBB.

11. The apparatus of claim 1, wherein the data processing unit is configured to determine data on the glymphatic activity based on the data on the hydrodynamics of water within the cranium and the physiological model.

12. The apparatus of claim 1, wherein the data processing unit is configured to detect an opening of a blood-brain-barrier based on electric signals from the electroencephalographic electrode arrangement of the at least one sensing device.

13. The apparatus of claim 1 wherein the data processing unit is configured to determine an opening of a blood-brain-barrier based on signal of the capacitive sensor arrangement.

14. The apparatus of claim 1, wherein the data processing unit comprises:
one or more processors,
one or more memories including computer program code; and
the one or more memories and the computer program code are configured to, with the one or more processors, cause the data processing unit for measuring the intracranial dynamics at least to: receive the electric signals, determine the at least one of the following dynamics: the glymphatic system, the water within the cranium, the brain tissue movements and the intracranial pressure based on the electric signals, and output the at least one piece of the data on the dynamics through the user interface.

15. A method of measuring intracranial dynamics,
the method comprising performing at least one of the following by at least one sensing device:
measuring direct-current electroencephalographic signals from the brain with an electroencephalographic electrode arrangement in electric contact with skin of a cranium,
directing optic radiation toward the brain, and receiving the optic radiation reflected and/or scattered therefrom with an optic measurement arrangement, and
measuring electric potential signals of the brain with a capacitive sensor arrangement in proximity without having an electric contact to the skin of the cranium; and
determining, by a data processing arrangement, based on electric signals from the at least one sensing device data on at least one of the following dynamics:
glymphatic system, water within the cranium, brain tissue movements, water and/or electrolyte movements and intracranial pressure based on at least one electric signal from the at least one sensing device; and
outputting the at least one piece of the data on the dynamics through a user interface.
